(19) United States
(12) Patent Application Publication
(10) Pub. No.: US 2025/0099222 A1
(43) Pub. Date: Mar. 27, 2025

(54) ARTIFICIAL BLOOD VESSEL AND MANUFACTURING METHOD FOR ARTIFICIAL BLOOD VESSEL

(71) Applicant: HI-LEX CORPORATION, Hyogo (JP)

(72) Inventor: Shinsaku KOARASHI, Hyogo (JP)

(21) Appl. No.: 18/832,204

(22) PCT Filed: Feb. 7, 2023
(86) PCT No.: PCT/JP2023/003899

§ 371 Date: Jul. 23, 2024

(30) Foreign Application Priority Data
Feb. 7, 2022 (JP) 2022-017487

(51) Int. Cl. A61F 2/06

(52) U.S. Cl. CPC A61F 2/06; A61F 2230/0069; A61F 2240/001; A61F 2250/0014

(57) ABSTRACT

In an artificial blood vessel of the present invention, the artificial blood vessel has a warp yarn 1 extending along the axial direction and a weft yarn 2 extending along the circumferential direction of the artificial blood vessel VE, and it has a structure in which the warp yarn 1 extends in a wavy shape when the artificial blood vessel VE is viewed in the radial direction such that positions of the warp yarn 1 at top parts Mt1, Mt2 of a pair of mountain parts M adjacent in the axial direction match in the circumferential direction and a position of the warp yarn 1 at a bottom part Vb of a valley part V between the pair of mountain parts M is offset in the circumferential direction relative to positions of the warp yarn 1 at the top parts Mt1, Mt2 of the pair of mountain parts M.

### TECHNICAL FIELD

The present invention relates to an artificial blood vessel and a manufacturing method for an artificial blood vessel.

### BACKGROUND ART

An artificial blood vessel is used, for example, for replacing a pathological living blood vessel. In accordance with the treatment site and the like, the artificial blood vessel is cut to a predetermined size and shape, sutured to a blood vessel in the human body, and the like, and used.

Since the artificial blood vessel may be bent and placed in the human body, it is preferable that the artificial blood vessel is flexible and is difficult to buckle when the artificial blood vessel is bent. To realize such performance, an artificial blood vessel with a pleated structure, in which a plurality of mountains and valleys are continuously provided in the axial direction of the artificial blood vessel, is known as shown in Patent document 1, for example. In Patent document 1, the artificial blood vessel with the pleated structure is formed by performing a predetermined processing on a tubular structural body without the pleated structure to. Specifically, a round bar with a spiral wire being wound around the round bar is inserted inside of the tubular structural body and another wire is spirally wound around outside of the tubular structural body. In this way, by firing the tubular structural body while the tubular structural body is constrained with a force applied thereon from outside by the wire, the tubular structural body becomes the artificial blood vessel with the pleated structure.

### PRIOR ART DOCUMENT

### Patent Document

Patent Document 1: JP S63-54171 A

### SUMMARY OF THE INVENTION

### Problem to be Solved by the Invention

In Patent document 1, the tubular structural body is composed of torsion lace, warp knit, and the like, but the tubular structural body may also be composed of a weaving structure with a warp yarn and a weft yarn. In the artificial blood vessel with a weaving structure, there is a need for an artificial blood vessel that is less likely to fray in the cut weft yarn when the artificial blood vessel is cut at an angle, for example.

Then, an object of the present invention is to provide an artificial blood vessel which is less likely to fray when the artificial blood vessel is cut, and a manufacturing method for the artificial blood vessel.

### Means to Solve the Problem

An artificial blood vessel of the present invention is an artificial blood vessel in which mountain parts and valley parts are alternately formed in an axial direction, wherein the artificial blood vessel has a warp yarn extending along the axial direction and a weft yarn extending along a circumferential direction of the artificial blood vessel, and wherein the warp yarn extends in a wavy shape when the artificial blood vessel is viewed in a radial direction of the artificial blood vessel such that positions of the warp yarn at top parts of a pair of mountain parts adjacent in the axial direction match in the circumferential direction and a position of the warp yarn at a bottom part of a valley part between the pair of mountain parts is offset in the circumferential direction relative to positions of the warp yarn at the top parts of the pair of mountain parts.

Moreover, a manufacturing method for an artificial blood vessel of the present invention is a manufacturing method for the above artificial blood vessel, the manufacturing method comprising the steps of: preparing a tubular body configured by a weaving structure of the warp yarn and the weft yarn; arranging the tubular body outside of a core material for molding, wherein the core material for molding has convex parts and concave parts corresponding to the mountain parts and the valley parts, respectively; in a state in which the tubular body is arranged outside of the core material for molding, winding a winding member onto outside of the tubular body around a part of the tubular body in the circumferential direction along the concave parts of the core material for molding; in a state in which the winding member is wound around the tubular body, relatively rotating, for a predetermined amount, a side in the axial direction of the tubular body, on which side the winding member is not wound around, relative to a side on which the winding member is wound around; and firing the tubular body in which the mountain parts and the valley parts are formed by the winding member.

### Effects of the Invention

According to the artificial blood vessel, and a manufacturing method for the artificial blood vessel of the present invention, it is possible to provide an artificial blood vessel which is less likely to fray when the artificial blood vessel is cut.

### BRIEF DESCRIPTION OF THE DRAWINGS

FIG. 1 is a side view of an artificial blood vessel of one embodiment of the present invention.

FIG. 2 is a partially enlarged view of a region II of FIG. 1.

FIG. 3 is a weaving structure diagram showing one example of a weaving structure of a base material used for the artificial blood vessel of FIG. 1.

FIG. 4 is a schematic enlarged view when the artificial blood vessel of FIG. 1 is viewed in the radial direction.

FIG. 5 is a schematic view showing a state in which a tubular body is arranged outside of a core material for molding and a winding member is partially wound around outside of the tubular body to form mountain parts and valley parts in the artificial blood vessel.

FIG. 6 is a schematic view of the tubular body when the tubular body is viewed in the axial direction in a state in which the tubular body is arranged outside of the core material for molding.

### EMBODIMENT FOR CARRYING OUT THE INVENTION

Below, an artificial blood vessel, and a manufacturing method for an artificial blood vessel of one embodiment of the present invention will be described with reference to the drawings. Besides, the embodiment shown below is merely one example, so that the artificial blood vessel, and the manufacturing method for an artificial blood vessel of the present invention are not limited to the embodiment below.

Besides, in the present specification, expressions “perpendicular to A” and similar thereto refer to not only a direction completely perpendicular to A, but refer to include a direction substantially perpendicular to A. Moreover, in the present specification, the expressions “parallel to B” and similar thereto refer not only to a direction completely parallel to B, but refer to include a direction substantially parallel to B. Furthermore, in the present specification, the expressions “C shape” and similar thereto refer not only to a complete C shape, but refer to include a shape that reminds the C shape in appearance (a substantially C shape).

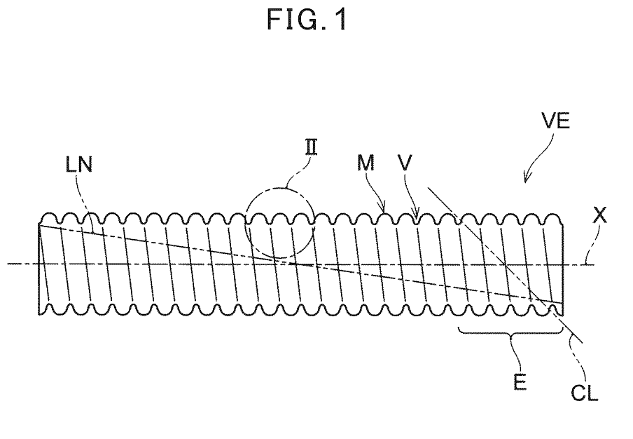

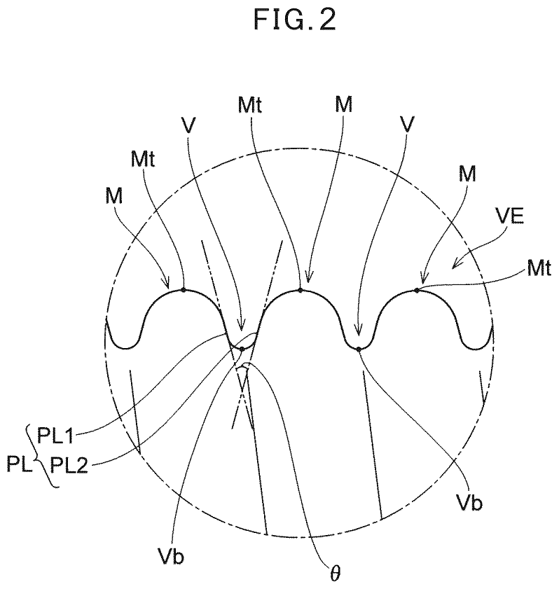

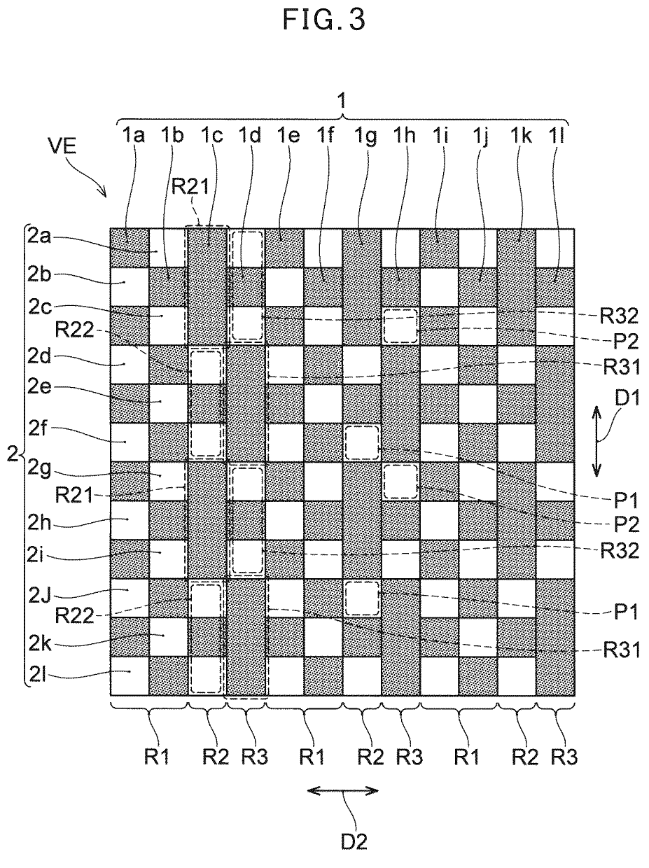

FIG. 1 is a side view of the artificial blood vessel of one embodiment of the present invention. FIG. 2 is a partially enlarged view of a region II of the artificial blood vessel of FIG. 1. FIG. 3 is a weaving structure diagram showing one example of a weaving structure of a base material used for the artificial blood vessel of FIG. 1.

The artificial blood vessel is used, such as, for example, for replacing a pathological living blood vessel and bypassing the living blood vessel. As shown in FIGS. 1 and 2, in an artificial blood vessel VE of the present invention, mountain parts M and valley parts V are alternately formed in an axial direction X (see FIG. 1). By the configuration in which the mountain parts M and the valley parts V are alternately formed in the artificial blood vessel VE, the artificial blood vessel becomes flexible and is not easy to kink when the artificial blood vessel VE is bent. The shape of the artificial blood vessel VE is not limited as long as the mountain parts M and the valley parts V are formed in the artificial blood vessel VE. However, in the present embodiment, the artificial blood vessel VE is formed in a shape of a circular tube in which the mountain parts M and the valley parts V are spirally formed.

The diameter of the artificial blood vessel VE may be changed in accordance with the site at which the artificial blood vessel is used, and the like, and is not limited. For example, the artificial blood vessel VE may be an artificial blood vessel with a large diameter having an inner diameter of 10 mm or more (for a thoracoabdominal aorta), an artificial blood vessel with a medium diameter having an inner diameter of 6 mm or more and less than 10 mm, such as 6 mm and 8 mm (for arteries in lower limb, neck, and axillary regions), or an artificial blood vessel with a small diameter having an inner diameter of less than 6 mm. The thickness of the artificial blood vessel VE is appropriately changed in accordance with the inner diameter and the length of the artificial blood vessel to be used, and is not limited. For example, the thickness of the artificial blood vessel VE may be 0.1 mm to 2 mm.

The length of the artificial blood vessel VE in the axial direction X may be changed in accordance with the site at which the artificial blood vessel is used, and the like, and is not limited. For example, the length of the artificial blood vessel VE in the axial direction X may be 100 mm to 1000 mm. It should be noted that, when the artificial blood vessel VE is implanted to a desired site, the artificial blood vessel VE is cut to a predetermined length and used by a physician and the like. The artificial blood vessel VE may be cut perpendicularly to the axial direction X or may be diagonally cut in an inclined manner at a predetermined angle with respect to the axial direction X depending on the site to which the artificial blood vessel is implanted (see a chain double-dashed line CL of FIG. 1, for example).

The number of mountain parts M (or valley parts V) (the number of pleats) in the artificial blood vessel VE is not limited, and may be appropriately set in accordance with the kink resistance performance required. For example, in a case of the artificial blood vessel VE with the outer diameter of 15 mm, the number of mountain parts M (the number of pleats) in the artificial blood vessel VE may be 20 to 70, preferably 25 to 35 for each 100 mm in length in the axial direction X. The interval (the pitch) in the axial direction X between a top part Mt (see FIG. 2) of a mountain part M of the artificial blood vessel VE and a top part Mt of an adjacent mountain part M of the artificial blood vessel VE is not limited, and the interval may be 10% to 30%, preferably 15% to 25% of the outer diameter (the outer diameter at the top part Mt of the mountain part M) of the artificial blood vessel VE, for example. The depth from the top part Mt of the mountain part M to a bottom part Vb of a valley part V (see FIG. 2) is not limited, and the depth may be 5% to 20%, preferably 5% to 15% of the outer diameter of the artificial blood vessel VE, for example.

In the present embodiment, the curvature at the top part Mt of the mountain part M is less than the curvature at the bottom part Vb of the valley part V (In the present embodiment, the curvature radius at the top part Mt of the mountain part M is greater than the curvature radius at the bottom part Vb of the valley part V.) It should be noted that “the curvature at the top part Mt of the mountain part M is less than the curvature at the bottom part Vb of the valley part V” means that the degree of curvature along the axial direction X at the top part Mt of the mountain part M is less than the degree of curvature along the axial direction X at the bottom part Vb of the valley part V (the curve of the mountain part M is more gradual than the curve of the valley part V), and the mountain part M and the valley part V do not have to form a complete arc plane. In a case that the curvature at the top part Mt of the mountain part M is less than the curvature at the bottom part Vb of the valley part V, when an external force is applied to the artificial blood vessel VE, stress concentrates at the valley part V. Therefore, the artificial blood vessel VE is likely to curve starting from the valley part V. The curvature of the mountain part M and the valley part V is not limited. For example, the curvature radius of the top part Mt of the mountain part M may be 5% to 8% of the diameter of the artificial blood vessel VE (and greater than the curvature radius of the bottom part Vb of the valley part V). Moreover, the curvature radius of the bottom part Vb of the valley part V may be 2% to 3% of the diameter of the artificial blood vessel VE (and greater than the curvature radius of the bottom part Vb of the valley part V). In a case that the artificial blood vessel VE is likely to curve, it is difficult for the curved artificial blood vessel VE to return to its original state and it is possible to reduce the load on the site connecting the artificial blood vessel VE to a blood vessel and the like.

A curved part at the top part Mt of the mountain part M and a curved part at the bottom part Vb of the valley part V may be connected by a planar part PL (see FIG. 2). This allows for improved flexibility and kink resistance compared to a case of directly connecting the curved parts. An angle θ formed by a planar part PL1 on one side and a planar part PL2 on the other side may be 20° to 40°, preferably 30°, and the angle θ formed by the planar part PL1 on one side and the planar par tPL2 on the other side may be appropriately set in accordance with the diameter of the artificial blood vessel, the height of the mountain part, the height of the valley part, the pitch, and the like.

Next, a configuration of a base material composing the artificial blood vessel VE will be described.

In the present embodiment, the artificial blood vessel VE is formed of a weaving structure of fibers. In the present embodiment, as shown in FIG. 3, the artificial blood vessel VE has warp yarns 1a to 1I (below, collectively called warp yarn 1) extending along the axial direction X (up-down direction in FIG. 3) and weft yarns 2a to 2I (below, collectively called weft yarn 2) extending along the circumferential direction (left-right direction in FIG. 3) of the artificial blood vessel VE. More specifically, as shown in FIG. 3, the artificial blood vessel VE has the plurality of warp yarns 1a to 1I and the plurality of weft yarns 2a to 2l, and has a weaving structure in which the warp yarns 1 and the weft yarns 2 are interlaced. In FIG. 3, the warp yarn 1 extends in the up-down direction, and the extending direction of the warp yarn 1 (the axial direction X of the artificial blood vessel VE) is called D1. Moreover, in FIG. 3, the weft yarn 2 extends in the left-right direction, and the extending direction of the weft yarn 2 (the circumferential direction of the artificial blood vessel VE) is called D2. In FIG. 3, what is shown in black (a portion shown with dots) is a portion in which the warp yarn 1 extends to the outer surface of the artificial blood vessel VE and what is shown in white is a portion in which the weft yarn 2 extends to the outer surface of the artificial blood vessel VE. A loom for manufacturing the artificial blood vessel VE is not limited.

In the present embodiment, the artificial blood vessel VE has a first region R1 in which the warp yarn 1 and the weft yarn 2 are woven in a plain weave, as shown in FIG. 3. Moreover, the artificial blood vessel VE has a second region R2 having a first portion on the second region side R21 in which the warp yarn 1 crosses over a plurality of weft yarns 2 on one surface of the artificial blood vessel VE (the outer surface of the artificial blood vessel VE in the present embodiment), and a second portion on the second region side R22 in which the warp yarn 1 extends so as to cross over one weft yarn 2 on one surface of the artificial blood vessel VE. Furthermore, the artificial blood vessel VE has a third region R3 having a first portion on the third region side R31 in which the warp yarn 1 crosses over a plurality of weft yarns 2 on one surface of the artificial blood vessel VE (the outer surface of the artificial blood vessel VE in the present embodiment) and a second portion on the third region side R32 in which the warp yarn 1 extends so as to cross over one weft yarn 2 on one surface of the artificial blood vessel VE. The first region R1, the second region R2, and the third region R3 are alternately formed in the extending direction D2 of the weft yarn 2, as shown in FIG. 3. That is, the first region R1, the second region R2, and the third region R3 are repeatedly arranged in this order in the extending direction D2 of the weft yarn 2. The first portion on the second region side R21 is adjacent to the second portion on the third region side R32 in the extending direction D2 of the weft yarn 2, and the second portion on the second region side R22 is adjacent to the first portion on the third region side R31 in the extending direction D2 of the weft yarn 2. In the present embodiment, the warp yarn 1 is composed of a multifilament yarn. In a case that the artificial blood vessel VE of the present embodiment has the above-described configuration, the warp yarn 1 composed of a multifilament yarn, which extends long without being restrained in the first portion on the second region side R21 or the first portion on the third region side R31, spreads toward the first region R1 woven in a plain weave (and in a direction perpendicular to one surface of the artificial blood vessel VE, namely, toward front direction of a paper surface in FIG. 3), as will be described later. With this three-dimensional structure of the warp yarn 1, when blood seeps out from a gap between fibers generated in the first region R1 woven in a plain weave, the blood is suppressed from leaking out and retained in the three-dimensional structure. By coagulating the blood with being retained, blood leakage resistance can be improved. A configuration and a weaving structure of each part of the artificial blood vessel VE will be described below.

### Configuration of Warp Yarn

The warp yarn 1 is a fiber extending in one direction, among fibers constituting the artificial blood vessel VE. In the present embodiment, the warp yarn 1 is a fiber extending in an axial direction X of the artificial blood vessel VE. The warp yarn 1 is made of a material applicable to a fabric artificial blood vessel composed of a weaving structure of fibers. The material of the warp yarn 1 is not particularly limited as long as it is a material applicable to the fabric artificial blood vessel. For example, the material of the warp yarn 1 can be polyester, polytetrafluoroethylene, polyamide, or the like. Moreover, a composite material composed of two or more kinds of applicable materials having different properties such as a melting point and a degree of shrinkage may be used. For example, the composite material may be a synthetic fiber in which polyethylene terephthalate (PET) and polytrimethylene terephthalate (PTT), etc. are combined at a spinning stage to form one long filament having a spiral crimp. For example, when the composite material composed of two kinds of materials having different melting point and degree of shrinkage, which has a spiral crimp, is used as a material of the warp yarn 1, a three-dimensional structure composed of a warp yarn 1 which will be described later is easy to spread in the extending direction D2 of the weft yarn 2, further enhancing a performance of retaining blood, which can improve the blood leakage resistance.

Each of the warp yarns 1 may be a monofilament yarn or a multifilament yarn, but is composed of the multifilament yarn in the present embodiment. The fineness of the warp yarn 1 is not limited, but, in a case that the warp yarn 1 is the monofilament yarn, for example, the single yarn fineness of the warp yarn may be 15 dtex to 100 dtex, preferably 20 dtex to 75 dtex. Moreover, in a case that the warp yarn 1 is the multifilament yarn, the fineness of the warp yarn 1 may be, for example, 0.25 to 2.50 dtex, preferably 0.50 to 2.00 dtex, for a single yarn fineness of the warp yarn 1, and may be 2 to 2500 dtex, preferably 6 to 1600 dtex, more preferably 10 to 540 dtex, further preferably 30 to 200 dtex, for a total fineness of the warp yarn 1. When the single yarn fineness of the warp yarn 1 and the total fineness of the warp yarn 1 are within the above-described ranges, the warp yarn 1 of the second region R2 and the third region R3 can be satisfactorily spread toward the first region R1. Therefore, when blood seeps out from a gap in the first region R1 with the warp yarn 1 of the second region R2 and the third region R3, the blood is suppressed from leaking out, retained by the three-dimensional structure of the warp yarn 1, and coagulates in the retained state, which can improve the blood leakage resistance. It should be noted that the “single yarn fineness” is a fineness per single filament constituting the warp yarn 1, and the “total fineness” is a product of the single yarn fineness and the number of filaments constituting the warp yarn 1. The number of filament yarns (hereinafter referred to as the number of filaments) constituting one warp yarn is not particularly limited. For example, as will be described later, when the total number of filaments of the warp yarn 1 is 1.5 times or more the number of filaments per single weft yarn 2 and the number of warp yarn 1 crossing over a plurality of weft yarns 2 is one in the second region R2, the number of filaments per single warp yarn 1 may be 8 to 1000, preferably 12 to 800, more preferably 20 to 270, further preferably 60 to 100. As will be described later, when the number of filaments per single warp yarn 1 is 0.8 to 1.2 times the number of filaments per single weft yarn 1 and the number of warp yarn 1 crossing over a plurality of weft yarns 2 is two or more in the second region R2, the number of filaments per single warp yarn 1 may be 4 to 500, preferably 6 to 400, more preferably 10 to 135, further preferably 30 to 50.

The weft yarn 2 is a fiber extending in a direction intersecting with the warp yarn 1, among fibers constituting the artificial blood vessel VE. In the present embodiment, the weft yarn 2 is a fiber extending in a circumferential direction of the artificial blood vessel VE. The weft yarn 2 is made of a material applicable to a fabric artificial blood vessel composed of a weaving structure of fibers. The material of the weft yarn 2 is not particularly limited as long as it is a material applicable to the fabric artificial blood vessel. For example, the material of the weft yarn 2 can be polyester, polytetrafluoroethylene, polyamide, or the like.

Each of the weft yarns 2 may be a monofilament yarn or a multifilament yarn, but in the present embodiment, the weft yarn 2 is composed of a multifilament yarn. A fineness of the weft yarn 2 is not particularly limited, but for example, when the weft yarn 2 is a monofilament yarn, a single yarn fineness of the weft yarn 2 may be 15 to 100 dtex, preferably 20 to 75 dtex. Moreover, when each weft yarn 2 is composed of a multifilament yarn, for example, the single yarn fineness of the weft yarn 2 may be 0.25 to 2.50 dtex, preferably 0.50 to 2.00 dtex, and a total fineness of the weft yarn 2 may be 1 to 1250 dtex, preferably 3 to 800 dtex, more preferably 5 to 270 dtex, further preferably 15 to 100 dtex. It should be noted that the “single yarn fineness” is a fineness per single filament (monofilament or multifilament) constituting the weft yarn 2, and the “total fineness” is a product of a single yarn fineness and the number of filaments constituting the weft yarn 2. When the weft yarn 2 is composed of a multifilament yarn, the number of filament yarns constituting one weft yarn may be 4 to 500, preferably 6 to 400, more preferably 10 to 135, further preferably 30 to 50.

The first region R1 is a section where the warp yarn 1 and the weft yarn 2 are plain-woven. In FIG. 3, the first region R1 is a region where the warp yarns 1a, 1b, 1e, 1f, 1i, 1j and the weft yarn 2 (weft yarns 2a to 2l) intersect. The first region R1 improves a strength of the artificial blood vessel VE, particularly a tensile strength (in the axial direction X of the artificial blood vessel VE). The first region R1 extends along the extending direction D1 of the warp yarn 1 and extends in the axial direction X of the artificial blood vessel VE. Moreover, a plurality of first regions R1 are arranged apart from each other at a predetermined interval in the extending direction D2 of the weft yarn 2. The second region R2 and the third region R3 are arranged between one first region R1 and the other first region R1 in the extending direction D2 of the weft yarn 2.

In the present embodiment, as shown in FIG. 1, two warp yarns 1a and 1b (the warp yarns 1e, 1f or the warp yarns 1i, 1j) and a plurality of weft yarns 2a to 2l (and weft yarns not shown) are plain-woven in the first region R1. The number of warp yarns 1 provided in one first region R1 can be 2 to 4, preferably 2 to 3, more preferably 2. It should be noted that, in the present specification, in a case that the warp yarn 1 is the multifilament yarn, the term “the number of warp yarns” refers to, with the warp yarn 1 composed of a plurality of filament yarns to be bundled being as one, the number of warp yarns 1 in which the filament yarns are bundled, not the number of filaments constituting multifilament yarns. When the number of warp yarn 1 is within the above-mentioned ranges, an uncovered area of the first region R1, which is not covered by the warp yarn 1 of the first portion on the second region side R21 and the warp yarn 1 of the first portion on the third region side R31, can be reduced. Therefore, the first region R1 in plain weave becomes easily covered three-dimensionally with the warp yarn 1 of the first portion on the second region side R21 and the warp yarn 1 of the first portion on the third region side R31. When blood seeps out from the first region R1, the blood is retained by the three-dimensional structure of the warp yarn 1 of the first portion on the second region side R21 and the warp yarn 1 of the first portion on the third region side R31 and coagulates in the retained state. Therefore, an amount of blood leakage from the artificial blood vessel VE can be reduced. Moreover, in the artificial blood vessel VE, a ratio of the number of warp yarn 1 in the first region R1 to the total number of warp yarn 1 arranged in the extending direction D2 of the weft yarn 2 in the first region R1 to the third region R3 (the number of warp yarn in the first region R1/the total number of warp yarn) is not particularly limited, but can be, for example, 0.2 to 0.4 (⅓ in the present embodiment). When the number of warp yarn 1 in the first region R1 and the ratio of the number of warp yarn 1 are within the above-described ranges, the amount of blood leakage from the artificial blood vessel VE can be reduced while increasing the strength of the artificial blood vessel VE.

The second region R2 has a first portion on the second region side R21 in which the warp yarn 1 crosses over a plurality of weft yarns 2 and a second portion on the second region side R22 in which the warp yarn 1 extends so as to cross over one weft yarn 2. As shown in FIG. 3, the first portion on the second region side R21 and the second portion on the second region side R22 are alternately provided in the extending direction D1 of the warp yarn 1. Since the second region R2 has the first portion on the second region side R21 and the second portion on the second region side R22, the artificial blood vessel VE can be made more flexible as compared with the artificial blood vessel VE, all region of which has a plain weave structure. It should be noted that a portion of the warp yarn 1c, which portion is provided in the second region R2, may be composed of single warp yarn or may be composed of a plurality of warp yarns. The number of warp yarn 1 provided in the second region R2 may be, for example, 1 to 4, preferably 2 to 3, more preferably 2.

The first portion on the second region side R21 is a portion woven so that the warp yarn 1 has a portion crossing over a plurality of weft yarns 2. In the present embodiment, the warp yarns 1c, 1g, 1k, etc. cross over the plurality of weft yarns 2. In the first portion on the second region side R21, the warp yarn 1 crosses over the plurality of weft yarns 2, so that the artificial blood vessel VE becomes more flexible in that portion than in the plain weave structure. Moreover, in a case that the warp yarn 1 of the first portion on the second region side R21 is composed of a multifilament yarn, both ends of the first portion on the second region side R21 in the extending direction D1 of the warp yarn 1 become in a state of being bound by the weft yarns 2 of the second portion on the second region side R22 (see the portion P1 in FIG. 3). In that case, the first portion on the second region side R21 of the warp yarn 1, which is composed of the multifilament yarn being bound at the both ends thereof, forms a three-dimensional structure spreading in the extending direction D2 of the weft yarn 2 at a center portion of the first portion on the second region side R21 in the extending direction D1 (besides, this three-dimensional structure also spreads in the left-right direction and in the direction toward the front of the paper surface in FIG. 3). Thus, the first region R1 with the plain weave structure adjacent to the first portion on the second region side R21 in the extending direction D2 of the weft yarn 2 is partially covered with the spread multifilament yarn of the first portion on the second region side R21. With this three-dimensional structure of the warp yarn 1, when blood seeps out from a gap between fibers generated in the plain-woven first region R1, the seeping blood is retained in a gap between filaments of the three-dimensional structure composed of the multifilament. As a result, the blood coagulates in the retained state, so that the blood leakage resistance can be improved. Moreover, in the present embodiment, the second portion on the third region side R32 adjacent to the first portion on the second region side R21 in the extending direction D2 of the weft yarn 2 is also partially covered with the spread multifilament yarn of the first portion on the second region side R21. As a result, a gap generated in the second portion on the third region side R32 is also covered with the multifilament yarn of the first portion on the second region side R21, so that the blood in the artificial blood vessel VE becomes less likely to leak out to the outside.

In the first portion on the second region side R21 (from a portion where the warp yarn 1 exits from the other surface of the artificial blood vessel VE to one surface of the artificial blood vessel VE (the surface shown in FIG. 3), to a portion where the warp yarn 1 enters into the other surface), the number of weft yarns 2 which the warp yarn 1 crosses over is not particularly limited, but can be, for example, 2 to 5, preferably 3 to 4, more preferably 3 (the state shown in FIG. 3). When the number of warp yarn 2 which the warp yarn 1 crosses over is within the above-described ranges, in the first portion on the second region side R21, the multifilament yarn of the warp yarn 1 is easy to be spread in the extending direction D2 of the weft yarn 2, and the strength of the artificial blood vessel VE can be maintained at a predetermined level.

In the first portion on the second region side R21, the number of warp yarn 1 constituting the first portion on the second region side R21 is not particularly limited as long as the warp yarn 1 has a portion crossing over a plurality of weft yarns 2. For example, the first portion on the second region side R21 (the second region R2) may be composed of a plurality of (two) warp yarns (each of the warp yarns 1c, 1g, 1k is composed of a plurality of warp yarns). Moreover, the first portion on the second region side R21 (the second region R2) may have at least one warp yarn 1 extending so as to cross over (only) one weft yarn 2 and at least one warp yarn 1 crossing over a plurality of weft yarns 2.

The second portion on the second region side R22 is a portion woven so that the warp yarn 1 crosses over only one weft yarn 2 (the warp yarn 1 does not cross over a plurality of weft yarns 2 from a portion where it exits from the other surface of the artificial blood vessel VE to one surface of the artificial blood vessel VE (the surface shown in FIG. 3) to a portion where it enters into the other surface). The second portion on the second region side R22 is set to have the same degree of length as the length of the first portion on the second region side R21 in the extending direction D1 of the warp yarn 1. That is, the number of weft yarn 2 in the first portion on the second region side R21 (three weft yarns 2 in FIG. 3) is equal to the number of weft yarn 2 in the second portion on the second region side R22 (three weft yarns 2 in FIG. 3).

The third region R3 has a first portion on the third region side R31 in which the warp yarn 1 crosses over a plurality of weft yarns 2 and a second portion on the third region side R32 in which the warp yarn 1 extends so as to cross over one weft yarn 2. As shown in FIG. 3, the first portion on the third region side R31 and the second portion on the third region side R32 are alternately provided in the extending direction D1 of the warp yarn 1. Since the third region R3 has the first portion on the third region side R31 and the second portion on the third region side R32, the artificial blood vessel VE can be made more flexible as compared with the artificial blood vessel VE, all region of which has a plain weave structure. It should be noted that a portion of the warp yarn 1d, which portion is provided in the third region R3, may be composed of single warp yarn or may be composed of a plurality of warp yarns. The number of warp yarn 1 provided in the third region R3 may be, for example, 1 to 4, preferably 2 to 3, more preferably 2.

The first portion on the third region side R31 is a portion woven so that the warp yarn 1 has a portion crossing over a plurality of weft yarns 2. In the present embodiment, the warp yarns 1d, 1h, 1l, etc. cross over the plurality of weft yarns 2. In the first portion on the third region side R31, the warp yarn 1 crosses over the plurality of weft yarns 2, so that the artificial blood vessel VE becomes more flexible in that portion than in the plain weave structure. Moreover, in a case that the warp yarn 1 of the first portion on the third region side R31 is composed of a multifilament yarn, both ends of the first portion on the third region side R31 in the extending direction D1 of the warp yarn 1 become in a state of being bound by the weft yarns 2 of the second portion on the third region side R32 (see the portion P2 in FIG. 3). In that case, the first portion on the third region side R31 of the warp yarn 1, which is composed of the multifilament yarn being bound at the both ends thereof, forms a three-dimensional structure spreading in the extending direction D2 of the weft yarn 2 at a center portion of the first portion on the third region side R31 in the extending direction D1 (besides, this three-dimensional structure also spreads in the left-right direction and in the direction toward the front of the paper surface in FIG. 3). Thus, the first region R1 with the plain weave structure adjacent to the first portion on the third region side R31 in the extending direction D2 of the weft yarn 2 is partially covered with the spread multifilament yarn of the first portion on the third region side R31. With this three-dimensional structure of the warp yarn 1, when blood seeps out from a gap between fibers generated in the plain-woven first region R1, the seeping blood is retained in a gap between filaments of the three-dimensional structure composed of the multifilament. As a result, the blood coagulates in the retained state, so that the blood leakage resistance can be improved. Moreover, in the present embodiment, the second portion on the second region side R22 adjacent to the first portion on the third region side R31 in the extending direction D2 of the weft yarn 2 is also partially covered with the spread multifilament yarn of the first portion on the third region side R31. As a result, a gap generated in the second portion on the second region side R22 is also covered with the multifilament yarn of the first portion on the third region side R31, so that the blood in the artificial blood vessel VE becomes less likely to leak out to the outside.

In the first portion on the third region side R31 (from a portion where the warp yarn 1 exits from the other surface of the artificial blood vessel VE to one surface of the artificial blood vessel VE (the surface shown in FIG. 3), to a portion where the warp yarn 1 enters into the other surface), the number of weft yarn 2 which the warp yarn 1 crosses over is not particularly limited, but can be, for example, 2 to 5, preferably 3 to 4, more preferably 3 (the state shown in FIG. 3). When the number of warp yarns 2 which the warp yarn 1 crosses over is within the above-described ranges, in the first portion on the third region side R31, the multifilament yarn of the warp yarn 1 is easy to be spread in the extending direction D2 of the weft yarn 2, and the strength of the artificial blood vessel VE can be maintained at a predetermined level.

In the first portion on the third region side R31, the number of warp yarn 1 constituting the first portion on the third region side R31 is not particularly limited as long as the warp yarn 1 has a portion crossing over a plurality of weft yarns 2. For example, the first portion on the third region side R31 (the third region R3) may be composed of a plurality of (two) warp yarns (each of the warp yarns 1d, 1h, 1l is composed of a plurality of warp yarns). Moreover, the first portion on the third region side R31 (the third region R3) may have at least one warp yarn 1 extending so as to cross over (only) one weft yarn 2 and at least one warp yarn 1 crossing over a plurality of weft yarns 2.

The second portion on the third region side R32 is a portion woven so that the warp yarn 1 crosses over only one weft yarn 2 (the warp yarn 1 does not cross over a plurality of weft yarns 2 from a portion where it exits from the other surface of the artificial blood vessel VE to one surface of the artificial blood vessel VE (the surface shown in FIG. 3) to a portion where it enters into the other surface). The second portion on the third region side R32 is set to have the same degree of length as the length of the first portion on the third region side R31 in the extending direction D1 of the warp yarn 1. That is, the number of weft yarn 2 in the first portion on the third region side R31 (three weft yarns 2 in FIG. 3) is the same as the number of weft yarn 2 in the second portion on the third region side R32 (three weft yarns 2 in FIG. 3).

It should be noted that the weaving structure of the artificial blood vessel VE is not limited to the above-described weaving structure. The artificial blood vessel VE may have, in whole or in part, a plain weaving structure, a twill weaving structure, a sateen weaving structure, or a combined structure of these weaving structures.

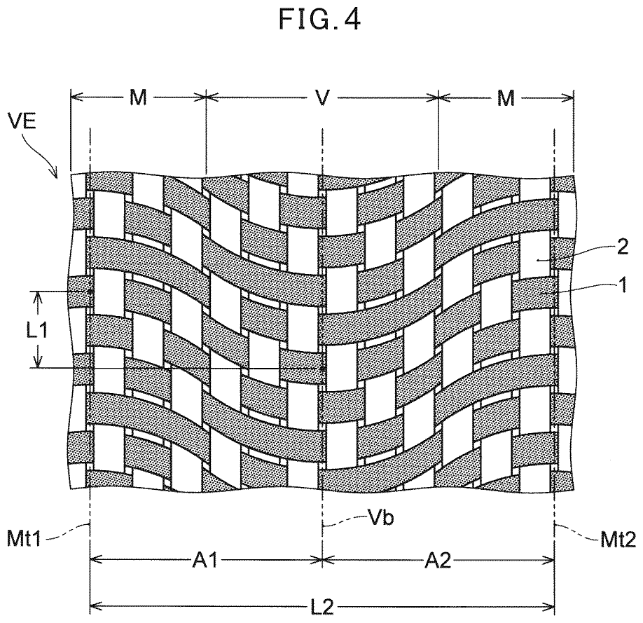

FIG. 4 is a schematic enlarged view when the artificial blood vessel VE is viewed in the radial direction, in other words, when the artificial blood vessel VE is viewed from outside toward the axis X of the artificial blood vessel VE (or when the artificial blood vessel VE is cut along the axis X and expanded). In FIG. 4, what is shown with dots is the warp yarn 1, while what is shown without dots is the weft yarn 2. Besides, FIG. 4 is shown in the same direction as the orientation of the artificial blood vessel VE in FIG. 1 and is shown in a direction in which the weaving structure diagram of FIG. 3 is rotated by 90°. In FIG. 4, top parts Mt1, Mt2 of the mountain parts M and the bottom part Vb of the valley part V of the artificial blood vessel VE are respectively shown in chain double-dashed lines. It should be noted that FIG. 4 is shown in deformed manner to make it possible to better understand how the warp yarn 1 extends, and a gap portion created between the warp yarn 1 and the weft yarn 2 in the schematic view of FIG. 4 is covered by the warp yarn 1 and the weft yarn 2 in the actual weaving structure of the artificial blood vessel VE.

In the present embodiment, as shown in FIG. 4, the warp yarn 1 extends in a wavy shape when the artificial blood vessel VE is viewed in the radial direction of the artificial blood vessel VE such that positions of the warp yarn 1 at top parts Mt1, Mt2 of a pair of mountain parts M adjacent in the axial direction X match in the circumferential direction and a position of the warp yarn 1 at a bottom part Vb of a valley part V between the pair of mountain parts M is offset in the circumferential direction relative to positions of the warp yarn 1 at the top parts Mt1, Mt2 of the pair of mountain parts M. Here, “positions at top parts Mt1, Mt2 of a pair of mountain parts M adjacent match in the circumferential direction” means that the positions in the circumferential direction at the top parts Mt1, Mt2 of the pair of mountain parts M adjacent do not have to be completely identical, so that the positions in the circumferential direction at the top parts Mt1, Mt2 of the pair of mountain parts M adjacent can be slightly offset as long as the warp yarn 1 extend in a wavy shape. It should be noted that in molding the artificial blood vessel, offsetting or deforming may occur at a part of the warp yarn 1 in the length direction, so that the positions in the circumferential direction at the top parts Mt1, Mt2 do not have to match over the whole length direction of the warp yarn 1. Moreover, “extend in a wavy shape when the artificial blood vessel VE is viewed in the radial direction” means that, when the one warp yarn 1 extending from one end to the other end in the axial direction X of the artificial blood vessel VE is viewed in the radial direction of the artificial blood vessel VE, the warp yarn 1 extends in a meandering manner so as to repeat displacement in the circumferential direction of the artificial blood vessel VE. Specifically, as shown in FIG. 4, a portion extending toward the bottom part Vb of the valley part V from the top part Mt1 of the mountain part M (see a region A1 in FIG. 4) of each warp yarn 1 of the artificial blood vessel VE extends, being displaced in one direction in the circumferential direction (as well as being displaced such that the position in the radial direction of the artificial blood vessel VE approaches the axis X). Moreover, a portion extending toward the top part Mt2 of the mountain part M from the bottom part Vb of the valley part V (see a region A2 in FIG. 4) of each warp yarn 1 of the artificial blood vessel VE extends, being displaced in the other direction in the circumferential direction (as well as being displaced such that the position in the radial direction of the artificial blood vessel VE separates from the axis X).

As described above, in a case that the warp yarn 1 extends in a wavy shape such that the position of the warp yarn 1 at the bottom part Vb of the valley part Vis offset in the circumferential direction relative to the positions of the warp yarn 1 at the top parts Mt1, Mt2 of the mountain parts M, the density of the warp yarn 1 (the amount of the warp yarn 1 for each unit area of the artificial blood vessel VE) is greater compared to a case in which the warp yarn extends linearly without being offset in the circumferential direction. In this case, the warp yarn 1 is well entangled with the weft yarn 2 and the weft yarn 2 is more strongly constrained by the warp yarn 1. Therefore, fraying of the weft yarn 2 can be suppressed in a case that the artificial blood vessel VE is cut, and the like. To describe specifically, for example, in a case that the warp yarn extends linearly when the artificial blood vessel is viewed in the radial direction (in a case that the warp yarn extends linearly to the left and right in FIG. 4), not extending in a wavy shape as shown in FIG. 4, the density of the warp yarn for each unit area is less. In this case, the entanglement between the warp yarn and the weft yarn loosens, so that the weft yarn is not strongly constrained by the warp yarn. Therefore, fraying of the weft yarn can occur when the artificial blood vessel VE is cut and the vicinity of the cut site of the artificial blood vessel is touched by a physician, etc., and the like. In particular, in a case that the artificial blood vessel VE is cut at an angle along a cutting line shown with the reference letter CL in FIG. 1, in an end region E of the artificial blood vessel VE, a half or more than a half in the circumferential direction of a plurality of weft yarns extending in a loop shape is removed. Therefore, in the end region E, the plurality of weft yarns is cut to a half or less of the length in the circumferential direction of the artificial blood vessel VE, are merely supported while being interlaced with the warp yarn, so that the weft yarn can easily fray. In this way, in a case that the weft yarn is not strongly constrained to the warp yarn, fraying may occur in the end region E between cutting the artificial blood vessel and suturing it to the blood vessel of the living body, and this fraying can be a cause of blood leakage after the artificial blood vessel is implanted to the living body. In the present embodiment, as described above, since the warp yarn 1 extends in a wavy shape such that the position of the warp yarn 1 at the bottom part Vb of the valley part Vis offset in the circumferential direction relative to the positions of the warp yarn 1 at the top parts Mt1, Mt2 of the mountain parts M, it is possible to strongly constrain the weft yarn 2 and to suppress fraying of the weft yarn 2 even when the artificial blood vessel VE is cut at an angle.

A position offset amount L1 (see FIG. 4) in the circumferential direction between the position of the warp yarn 1 at the bottom part Vb of the valley part V and the positions of the warp yarn 1 at the top parts Mt1, Mt2 of the mountain parts M are appropriately changed in accordance with the flexibility and kink resistance required for the artificial blood vessel. For example, the above-described position offset amount L1 may be 5% to 25%, more preferably 8% to 20% of an interval L2 (see FIG. 4) between the top parts Mt1, Mt2 of the mountain parts M.

Moreover, since the positions of the warp yarn 1 at the top parts Mt of the mountain parts M match in the circumferential direction of the artificial blood vessel VE, the warp yarn 1 extends in a wavy shape from one end to the other end of the artificial blood vessel VE and extends along the axis X of the artificial blood vessel VE as a whole. In a case that the warp yarn extends in an inclined manner relative to the axis X from one end to the other end of the artificial blood vessel (see the chain double-dashed line LN in FIG. 1), the artificial blood vessel twists around the axis X or bend with respect to the axis X in the natural state (unloaded state).

On the other hand, in the present embodiment, since the warp yarn 1 extends in a wavy shape from one end to the other end of the artificial blood vessel VE and extends along the axis X of the artificial blood vessel VE as a whole, it is suppressed that the artificial blood vessel VE twists around the axis X or bends with respect to the axis X in the natural state.

Furthermore, in the present embodiment, as described above, the artificial blood vessel VE alternately has, in the extending direction D2 of the weft yarn 2, a first region R1 in which the warp yarn 1 and the weft yarn 2 are woven in the plain weave, the second region R2 having the first portion on the second region side R21 and the second portion on the second region side R22, and the third region R3 having the first portion on the third region side R31 and the second portion on the third region side R32. The first portion on the second region side R21 is adjacent to the second portion on the third region side R32 in the extending direction D2 of the weft yarn 2. The second portion on the second region side R22 is adjacent to the first portion on the third region side R31 in the extending direction D2 of the weft yarn 2. The warp yarn 1 is composed of a multifilament yarn. In this case, the warp yarn 1 composed of the multifilament yarn is bundled by the weft yarn 2 at both end portions of a portion crossing over the plurality of weft yarns 2 (see portions P1, P2 in FIG. 3). The weft yarn 2 is subjected to a strong constraining force due to the reaction force, which is caused by spreading of the multifilament yarn of the warp yarn 1 bundled by the weft yarn 2. Therefore, the weft yarn 2 is constrained by the warp yarn 1, so that fraying of the weft yarn 2 is further suppressed.

Moreover, in a case of the weaving structure shown in FIG. 3, the multifilament yarn of the warp yarn 1 in the first portion on the second region side R21 spreads in the extending direction D2 of the weft yarn 2 and partially covers the first region R1 adjacent to the first portion on the second region side R21 to fill gaps (porosities) formed at four corners of an intersection part between the warp yarn 1 and the weft yarn 2 formed in the first region R1. Furthermore, the multifilament yarn of the warp yarn 1 in the first portion on the third region side R31 spreads in the extending direction D2 of the weft yarn 2 and partially covers the first region R1 adjacent to the first portion on the third region side R31 to cover the gaps (porosities) formed at the four corners of the intersection part between the warp yarn 1 and the weft yarn 2 formed in the first region R1 with multifilaments in the first portion on the second region side R21 and the first portion on the third region side R31. Therefore, when blood seeps out from a gap between fibers generated in the first region R1 woven in a plain weave, with the three-dimensional structure of the warp yarn 1, the seeping blood is retained in a gap between filaments having a three-dimensional structure composed of multifilaments, allowing for the blood to coagulate without flowing out. Thereby, the blood leakage resistance can be improved. In addition, in the present embodiment, the second portion on the third region side R32 adjacent to the first portion on the second region side R21 is partially covered with the multifilament yarn of the warp yarn 1 in the first portion on the second region side R21 to cover gaps (porosities) formed at an intersection part of the warp yarn 1 and the weft yarn 2 formed in the second portion on the third region side R32. Furthermore, the second portion on the second region side R22 adjacent to the first portion on the third region side R31 is partially covered with the multifilament yarn of the warp yarn 1 in the first portion on the third region side R31 to cover gaps (porosities) formed at an intersection part of the warp yarn 1 and the weft yarn 2 formed in the second portion on the second region side R22. Therefore, the blood in the artificial blood vessel becomes less likely to leak out to the outside through a gap between the second portion on the third region side R32 and the second portion on the second region side R22, improving the blood leakage resistance of the artificial blood vessel VE.

Furthermore, in the present embodiment, the first region R1 having a plain weave structure, and the second region R2 and the third region R3 each having a weave structure different from the plain weave structure are alternately formed in the extending direction D2 of the weft yarn 2. Therefore, a predetermined flexibility required for the artificial blood vessel VE can be obtained with the second region R2 and the third region R3, while securing a predetermined strength of the artificial blood vessel VE with the first region R1 provided at a predetermined interval in the extending direction D2 of the weft yarn 2. Therefore, in a case that the artificial blood vessel VE having a weaving structure shown in FIG. 3 is provided, in addition to improvement of the blood leakage resistance, both strength and flexibility required for the artificial blood vessel VE can be achieved.

Moreover, in the present embodiment, as shown in FIG. 1, the first portion on the second region side R21 and the first portion on the third region side R31 are configured to extend continuously in the extending direction D1 of the warp yarn 1 in a zigzag shape. In this case, the warp yarn 1 of the first portion on the second region side R21 and the warp yarn 1 of the first portion on the third region side R31, which are spread in the extending direction D2 of the weft yarn 2, do not interfere with each other, and the spread of the warp yarn 1 is not interrupted in the direction D1 of the warp yarn 1, so that absorbability of blood with the three-dimensional structure can be further enhanced.

It is preferable that an average width of the maximum spread of the warp yarn 1 in the extending direction D2 of the weft yarn 2 at the first portion on the second region side R21 and the first portion on the third region side R31 in the extending direction D2 of the weft yarn 2 is larger than an average width of the maximum spread of the warp yarn 1 at the first region R1 in the extending direction D2 of the weft yarn 2. In this case, gaps between the first region R1, the second portion on the second region side R22, and the second portion on the third region side R32 are covered in a large region with the warp yarn 1 of the first portion on the second region side R21 and the first portion on the third region side R31. Therefore, blood seeping out from the gaps between the first region R1, the second portion on the second region side R22, and the second portion on the third region side R32 is easy to be further retained, and the blood leakage resistance can be further improved. It should be noted that the average width of the maximum spread of the warp yarn 1 in the extending direction D2 of the weft yarn 2 at the first portion on the second region side R21 and the first portion on the third region side R31 is not particularly limited, but may be, for example, 2.0 to 4.0 times the average width of the maximum spread of the warp yarn 1 in the extending direction D2 of the weft yarn 2 at the region R1.

It should be noted that the “average width of the maximum spread of the warp yarn 1 of the first portion on the second region side R21 and the first portion on the third region side R31 in the extending direction D2 of the weft yarn 2” may be obtained by, for example, measuring a predetermined number m of (for example, 10 or more) widths Wa (not shown) of portions where spreads of the warp yarn 1 of the first portion on the second region side R21 and the first portion on the third region side R31 are maximized, in a predetermined area of the artificial blood vessel (for example, 1 mm×1 mm), to calculate an average value thereof ((Wa1+Wa2+ . . . Wam)/m).

Moreover, in the artificial blood vessel VE, the weft yarn 2 is composed of a multifilament yarn, and in the second region R2 and the third region R3, the total number of filaments of the warp yarn 1 crossing over the plurality of weft yarns 2 (the warp yarns 1c, 1d, 1g, 1h, 1k, 1l in FIG. 3) may be 1.5 times or more, preferably 1.5 to 3.0 times the number of filaments per single weft yarn 2. Here, “the total number of filaments of the warp yarn 1 crossing over the plurality of weft yarns 2” mean, in a case that the number of warp yarn 1 crossing over the plurality of weft yarns 2 is one in one second region R2 or one third region R3, the number of filaments for the one warp yarn, and in a case that the number of warp yarn 1 crossing over the plurality of weft yarns 2 is two or more (for example, two or three), the total number of filament yarns for the plurality of warp yarns 1 (the number of filaments constituting the one warp yarn 1 multiplied by 2 or 3 which is the number of warp yarn). When the total number of filaments of the warp yarn 1 crossing over the plurality of weft yarns 2 are larger than the number of filaments per single weft yarn 2, the multifilament yarn of the warp yarn 1 is easier to spread than the multifilament yarn of the weft yarn 2, and the blood leakage resistance can be further enhanced. That is, the warp yarn 1 having a larger total number of filaments than the weft yarn 2 is bound by the weft yarn 2 that is thinner (namely, has a smaller number of filaments) than the warp yarn 1, at both ends of the first portion on the second region side R21 and the first portion on the third region side R31, in the extending direction D1 of the warp yarn 1. As a result, with the warp yarn 1 being bound by the thin weft yarn 2, a strong pressure is applied to the warp yarn 1, and the warp yarn 1 becomes more easily spread in the extending direction D2 of the weft yarn 2. In addition, the strong pressure applied to the warp yarn 1 causes an even greater reaction force to be applied to the weft yarn 2, further improving the fraying prevention effect of the weft yarn 2. Furthermore, when the total number of filaments of the warp yarn 1 and the number of filaments per single weft yarn 2 are provided in the above-described ratios, the number of the weft yarn 2 becomes smaller than the number of filaments of the warp yarn 1, therefore, it becomes easy to close up the weft yarns 2 in the extending direction D1 of the warp yarn 1 in weaving an artificial blood vessel. Therefore, by closing up the weft yarns 2 in the extending direction D1 of the warp yarn 1, the gaps (porosities) formed at the intersection part between the warp yarn 1 and the weft yarn 2 can be reduced, and the blood leakage amount itself can be reduced. Therefore, the blood leakage resistance can be dramatically enhanced by the synergistic effect of the reduction of the blood leakage amount itself by facilitating the closing up of the weft yarns 2 and the absorbability of blood leaking with the three-dimensional structure of the warp yarn 1.

In the present embodiment, preferably, in each of the second region R2 and the third region R3, the number of warp yarn 1 crossing over the plurality of weft yarns 2 is configured to be one, and the number of filaments per single warp yarn 1 in the second region R2 and the third region R3 is configured to be 1.5 times or more, preferably 1.5 to 3 times the number of filaments per single weft yarn. Specifically, the number of filaments per single weft yarn 2 can be 4 to 500, and the number of filaments per single warp yarn 1 can be 8 to 1000. As a result, in the second region R2 and the third region R3, multifilament yarns constituting the warp yarn 1 crossing over the plurality of weft yarns 2 are bundled into one in each of the second region R2 and the third region R3, the number of which is larger than the number of filaments of the weft yarns 2. It should be noted that, in the second region R2 and the third region R3, configurations of the warp yarn 1 and the weft yarn 2 are not particularly limited to the above-mentioned configurations as long as the total number of filaments of the warp yarn 1 crossing over the plurality of weft yarns 2 is configured to be 1.5 times or more the number of filaments per single weft yarn 2. For example, in the second region R2 and the third region R3, the number of warp yarn 1 crossing over the plurality of warp yarns 2 may be two or more, and the number of filaments per single warp yarn 1 may be 0.8 to 1.2 times the number of filaments per single weft yarn 2 (preferably the same number of filaments). Also in this case, when the number of warp yarn 1 crossing over the plurality of weft yarns 2 is two or more, in the second region R2 and the third region R3, the total number of filaments of the warp yarn 1 crossing over the plurality of weft yarns 2 is larger than the number of filaments per single weft yarn 2. Therefore, the similar effect as the above-mentioned effect can be obtained.

Moreover, in the second region R2 and the third region R3, the number of filaments of the warp yarn 1 (for example, 1c, 1d, 1g, 1h, 1k, 1l) crossing over the plurality of weft yarns 2 may be larger than the number of filaments per single weft yarn 2 (for example, 1.5 to 3 times), and two warp yarns 1 may be provided in each of the second region R2 and the third region R3 (for example, each of 1c, 1d, 1g, 1h, 1k, 1l is composed of two warp yarns). These two warp yarns 1 each of which has filaments larger than the number of filaments per single weft yarn 2 are bundled with one weft yarn 2 having a smaller number of filaments. In this case, a reaction force applied from the warp yarn 1 to the one weft yarn 2 becomes larger than the reaction force applied in a case of bundling one warp yarn or in a case of bundling warp yarns having a smaller number of filaments per single warp yarn. Therefore, the above-described fraying prevention effect of the weft yarn 2 further improves. Furthermore, as mentioned above, in the second region R2 and the third region R3, the warp yarn 1 spreads in the extending direction D2 of the weft yarn 2 to cover the surface of the weft yarn 2. As a result, the weft yarn 2 becomes less likely to be exposed on the surface of the artificial blood vessel VE, and when a doctor or the like touches the artificial blood vessel VE, chances of touching the weft yarn 2 are reduced, so that the weft yarn 2 is suppressed from fraying from the cut portion of the artificial blood vessel VE.

Next, one example of a manufacturing method for the above-described artificial blood vessel VE will be explained. It should be noted that the manufacturing method below is merely one example, the artificial blood vessel VE can be manufactured by another manufacturing method, and the artificial blood vessel VE and the manufacturing method for the artificial blood vessel VE of the present invention are not limited by the explanations below.

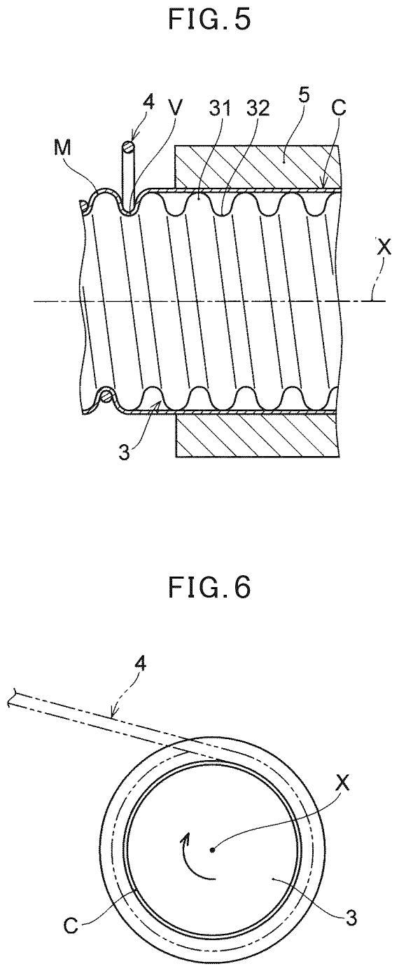

First, a tubular body C (see FIG. 5) configured by the weaving structure of the warp yarn 1 and the weft yarn 2, such as the above-described weaving structure, for example, is prepared. It should be noted that the “tubular body C” as referred to herein refers to a tubular base material in which the mountain parts M and the valley parts V shown in FIGS. 1 and 2 have not been formed yet (or the mountain parts M and the valley parts V are formed only in one part and the mountain parts M and the valley parts V have not been formed yet in the other part). For a forming method for the tubular body C, a known method to manufacture a tubular artificial blood vessel (an artificial blood vessel not having pleats) having a predetermined weaving structure can be adopted, so that the explanations will be omitted.

Next, the tubular body C is arranged outside of a core material for molding 3 (see FIG. 5) having convex parts 31 and concave parts 32 corresponding to the mountain parts M and the valley parts V, respectively. The core material for molding 3 has a size and shape corresponding to the artificial blood vessel VE having the mountain parts M and the valley parts V of a predetermined size and shape. In the present embodiment, the core material for molding 3 is configured so as to be covered by the tubular body C outside the core material for molding 3. The outer diameter of the convex part 31 of the core material for molding 3 is preferably the same as or less than the inner diameter of the tubular body C, but the outer diameter of the convex part 31 may be slightly greater than the inner diameter of the tubular body C. In the present embodiment, the core material for molding 3 is supported to a support (not shown) of a molding apparatus including the core material for molding 3 so as to be rotatable around the axis X.

When the tubular body C is arranged outside of the core material for molding 3, as shown in FIG. 5, in a state in which the tubular body C is arranged outside of the core material for molding 3, a winding member 4 is wound onto the outside of the tubular body C around a part of the tubular body C in the circumferential direction along the concave parts 32 of the core material for molding 3. The winding member 4 is a member to form parts corresponding to the valley parts V in the tubular body C by pressing the part of the tubular body C arranged outside of the core material for molding 3 against the concave part 32 of the core material for molding 3. While the winding member 4 is not particularly limited as long as the winding member 4 can form the valley parts V in the tubular body C, in the present embodiment, the winding member 4 may be configured to be a wire having a size that allows the wire to get in between a pair of convex parts 31 corresponding to a pair of mountain parts M. In the present embodiment, the winding member 4 is stretched in a tensioned state (see FIG. 6) and the core material for molding 3 is rotated around the axis X. Accordingly, the winding member 4 is spirally wound around the outer circumference of the tubular body C, forming the valley parts V in the tubular body C. It should be noted that the valley parts V may be formed in the tubular body C by moving the winding member 4 so as to be spirally wound around the core material for molding 3 without rotating the core material for molding 3.

In the above-described step of winding the winding member 4 onto the outside of the tubular body C, the valley parts V are formed in the tubular body C while the tubular body C is pressed against the core material for molding 3 by the winding member 4. At this time, since the winding member 4 is wound around the tubular body C while the tubular body C is pressed against by the winding member 4, the warp yarn 1 of the tubular body C is pulled in the circumferential direction by the winding member 4. Therefore, the warp yarn 1 is subjected to a force by the winding member 4 such that the warp yarn 1 is offset in the circumferential direction, and the warp yarn 1 is offset in the circumferential direction relative to an unpressed top part Mt of the mountain part M (see the region A1 in FIG. 4). At this time, in a case that the warp yarn 1 is offset (twisted) in the circumferential direction, the warp yarn 1 is pulled in the extending direction of the warp yarn 1 relative to when the warp yarn 1 extends substantially in parallel to the axis X. In this way, a gap between the warp yarn 1 and the weft yarn 2 is further packed, making it possible to further reduce the blood leaking out. Moreover, to form the valley parts V, the tubular body C is pressed toward the concave part 32 of the core material for molding 3 inwardly in the radial direction of the tubular body C by the winding member 4 from a state without any concave or convex along the axial direction X shown on the right in FIG. 5. At this time, the warp yarn 1 of the tubular body C is pulled in the extending direction of the warp yarn 1 while being deformed along the concave part 32. In this way, a gap between the warp yarn 1 and the weft yarn 2 is further packed, improving the blood leakage resistance performance.

Next, in a state in which the winding member 4 is wound around the tubular body C, a side in the axial direction X of the tubular body C, on which side the winding member 4 is not wound around (a portion on the right in FIG. 5) is relatively rotated for a predetermined amount relative to a side on which the winding member 4 is wound around (a portion on the left in FIG. 5). In the above-described step of winding the winding member 4, the position of the warp yarn 1 in the circumferential direction is offset between a top part Mt1 of the one mountain part M and a bottom part Vb of the valley part V adjacent to the top part Mt1 of the mountain part M (see the region A1 in FIG. 4), but with this rotating step, the tubular body C is rotated for a predetermined amount such that the position in the circumferential direction of the top part Mt2 of the other mountain part M being adjacent in the axial direction X to the one mountain part M matches the position in the circumferential direction of the top part Mt1 of the one mountain part M. More specifically, for example, the core material for molding 3 is rotated around the axis X at a rotation angle of 360° or less (for example,) 90° to wind the winding member 4 around the tubular body C for a predetermined amount, and then a side of the tubular body C, on which side the winding member 4 is not wound around, is slightly rotated such that the position in the circumferential direction of the top part Mt2 of the mountain part M matches the position in the circumferential direction of the top part Mt1 of the mountain part M. In this way, the positions in the circumferential direction of the top parts Mt1, Mt2 of the mountain parts M can be different from the position of the bottom part Vb of the valley part V, making it possible to form the warp yarn 1 in a wavy shape. The relative rotation of the tubular body C in a predetermined amount can be at a rotation angle such that the positions in the circumferential direction of the top parts Mt1, Mt2 of the pair of mountain parts M being adjacent in the axial direction X of the warp yarn 1 are substantially the same. The setting method of the “predetermined amount”, which is the rotation amount of the tubular body C, is not particularly limited. For example, an amount of position offset in the circumferential direction between the top part Mt of the mountain part M and the bottom part Vb of the valley part V of the warp yarn 1 when the winding member 4 is wound, for a predetermined length, around a tubular body C (sample) having a similar material and structure is calculated, and the rotation angle of the tubular body C that can eliminate the calculated amount of position offset can be defined as a predetermined rotation amount of the tubular body C. Moreover, a position offset amount in the circumferential direction between the top part Mt of the mountain part M and the bottom part Vb of the valley part V of the warp yarn 1 can be measured by a sensor and the like that can sense the position of the warp yarn 1 in the circumferential direction, and the rotation angle of the tubular body C that can eliminate the amount of position offset measured by the sensor and the like can be defined as a predetermined rotation amount of the tubular body C.

The method of relatively rotating the tubular body C for a predetermined amount is not particularly limited as long as a side in the axial direction X of the tubular body C, on which side the winding member 4 is not wound around, can be rotated relative to a side on which the winding member 4 is wound around. For example, as shown in FIG. 5, a holding part 5 that can hold a portion of the tubular body C, in which portion the winding member 4 is not wound around, can be provided further on the outside of the tubular body C arranged outside of the core material for molding 3. The holding part 5 is configured to rotate a side of the tubular body C, on which side the winding member 4 is not wound around, relative to a side on which the winding member 4 is wound around. By this relative rotation, the warp yarn 1 is twisted (in the region A2 in FIG. 4) in the direction opposite to a portion (see the region A1 in FIG. 4) of the warp yarn 1 that is twisted by the winding member 4 as shown in FIG. 4. Therefore, the top parts Mt1, Mt2 of the pair of adjacent mountain parts M can be in the same position in the circumferential direction. In a case that the wavy shape of the warp yarn 1 is formed in a twisted manner such that the warp yarn 1 is inclined relative to the axial direction X by the holding part 5 and the like, the entanglement between the warp yarn 1 and the weft yarn 2 becomes stronger, further improving the blood leakage resistance.

The step of winding the winding member 4 onto the outside of the tubular body C and the step of relatively rotating, for a predetermined amount, the side in the axial direction X of the tubular body C, on which side the winding member 4 is not wound around, relative to a side on which the winding member 4 is wound around are repeated until the winding member 4 is wound around over almost the entirety of the tubular body C in the axial direction X. When the winding member 4 is wound around over almost the entirety of the tubular body C in the axial direction X, the tubular body C in which the mountain parts M and the valley parts V are formed is fired by the winding member 4. The firing of the tubular body C is completed, the tubular body C is cooled, and the winding member 4 and the core material for molding 3 are removed, thereby the artificial blood vessel VE is completed.

In the artificial blood vessel VE manufactured by the above-described manufacturing method, as shown in FIG. 4, the warp yarn 1 extends in a wavy shape when the artificial blood vessel VE is viewed in the radial direction (when the artificial blood vessel VE is cut along the axial direction and expanded) such that the position of the warp yarn 1 at the bottom part Vb of the valley part V is offset in the circumferential direction relative to the positions at the top parts Mt of the mountain parts M. In this case, the density of the warp yarn 1 (the amount of the warp yarn 1 for each unit area of the artificial blood vessel VE) is greater compared to a case in which the warp yarn extends linearly without being offset in the circumferential direction, and the weft yarn 2 is more strongly constrained by the warp yarn 1. Therefore, fraying of the weft yarn 2 can be suppressed in a case that the artificial blood vessel VE is cut, and the like. Moreover, since the positions of the warp yarn 1 at the top parts Mt1, Mt2 of the mountain parts M match in the circumferential direction of the artificial blood vessel VE, the warp yarn 1 extends in a wavy shape from one end to the other end of the artificial blood vessel VE and extends along the axis X of the artificial blood vessel VE as a whole. Therefore, it is suppressed that the artificial blood vessel VE twists around the axis X or bends with respect to the axis X in the natural state. Furthermore, in the present embodiment, as described above, the artificial blood vessel VE alternately has, in the extending direction D2 of the weft yarn 2, a first region R1 in which the warp yarn 1 and the weft yarn 2 are woven in the plain weave, the second region R2 having the first portion on the second region side R21 and the second portion on the second region side R22, and the third region R3 having the first portion on the third region side R31 and the second portion on the third region side R32. The first portion on the second region side R21 is adjacent to the second portion on the third region side R32 in the extending direction D2 of the weft yarn 2. The second portion on the second region side R22 is adjacent to the first portion on the third region side R31 in the extending direction D2 of the weft yarn 2. The warp yarn 1 is composed of a multifilament yarn. In this case, the warp yarn 1 composed of the multifilament yarn is bundled by the weft yarns 2 at both end portions of a portion crossing over the plurality of weft yarns 2 and the weft yarns 2 are subjected to a strong constraining force due to the reaction force, which is caused by spreading of the multifilament yarn of the warp yarn 1 bundled by the weft yarns 2. Therefore, the weft yarns 2 are constrained by the warp yarn 1, so that fraying of the weft yarns 2 is further suppressed.

### REFERENCE SIGNS LIST

1, 1a, 1b, 1c, 1d, 1e, 1f, 1g, 1h, 1i, 1j, 1k, 1l WARP YARN
2, 2a, 2b, 2c, 2d, 2e, 2f, 2g, 2h, 2i, 2j, 2k, 2l WEFT YARN
3 CORE MATERIAL FOR MOLDING
31 CONVEX PART
32 CONCAVE PART
4 WINDING MEMBER
5 HOLDING PART
A1 REGION OF WARP YARN EXTENDING FROM TOP PART OF MOUNTAIN PART TOWARD BOTTOM PART OF VALLEY PART OF WARP YARN
A2 REGION OF WARP YARN EXTENDING FROM BOTTOM PART OF VALLEY PART TOWARD TOP PART OF MOUNTAIN PART OF WARP YARN
C TUBULAR BODY
CL CUTTING LINE
D1 EXTENDING DIRECTION OF WARP YARN
D2 EXTENDING DIRECTION OF WEFT YARN
E END REGION OF ARTIFICIAL BLOOD VESSEL
L1 POSITION OFFSET AMOUNT IN CIRCUMFERENTIAL DIRECTION BETWEEN BOTTOM PART OF VALLEY PART AND TOP PART OF MOUNTAIN PART
L2 INTERVAL BETWEEN TOP PARTS OF MOUNTAIN PARTS
LN VIRTUAL LINE IN A CASE THAT WARP YARN EXTENDS IN INCLINED MANNER RELATIVE TO THE AXIS FROM ONE END TO THE OTHER END OF ARTIFICIAL BLOOD VESSEL
M MOUNTAIN PART
Mt, Mt1, Mt2 TOP PART OF MOUNTAIN PART
P1 PORTION OF WEFT YARN BINDING BOTH ENDS OF FIRST PORTION ON THE SECOND REGION SIDE
P2 PORTION OF WEFT YARN BINDING BOTH ENDS OF FIRST PORTION ON THE THIRD REGION SIDE
PL, PL1, PL2 PLANNAR PART
R1 FIRST REGION
R2 SECOND REGION
R21 FIRST PORTION ON THE SECOND REGION SIDE
R22 SECOND PORTION ON THE SECOND REGION SIDE
R3 THIRD REGION
R31 FIRST PORTION ON THE THIRD REGION SIDE
R32 SECOND PORTION ON THE THIRD REGION SIDE
V VALLEY PART
Vb BOTTOM PART OF VALLEY PART
VE ARTIFICIAL BLOOD VESSEL
X AXIS
θ ANGLE FORMED BY PLANAR PART ON ONE SIDE AND PLANAR PART ON THE OTHER SIDE

1.-5. (canceled)

6. An artificial blood vessel in which mountain parts and valley parts are alternately formed in an axial direction,
wherein the artificial blood vessel has a warp yarn extending along the axial direction and a weft yarn extending along a circumferential direction of the artificial blood vessel, and
wherein the warp yarn extends in a wavy shape when the artificial blood vessel is viewed in a radial direction of the artificial blood vessel such that positions of the warp yarn at top parts of a pair of mountain parts adjacent in the axial direction match in the circumferential direction and a position of the warp yarn at a bottom part of a valley part between the pair of mountain parts is offset in the circumferential direction relative to positions of the warp yarn at the top parts of the pair of mountain parts.

7. The artificial blood vessel according to claim 6, wherein the artificial blood vessel alternately has, in an extending direction of the weft yarn,
a first region in which the warp yarn and the weft yarn are woven in a plain weave,
a second region having a first portion on the second region side in which the warp yarn crosses over a plurality of weft yarns on one surface of the artificial blood vessel and a second portion on the second region side in which the warp yarn extends so as to cross over one weft yarn on one surface of the artificial blood vessel, and
a third region having a first portion on the third region side in which the warp yarn crosses over a plurality of weft yarns on one surface of the artificial blood vessel and a second portion on the third region side in which the warp yarn extends so as to cross over one weft yarn on one surface of the artificial blood vessel,
wherein the first portion on the second region side is adjacent to the second portion on the third region side in the extending direction of the weft yarn, and the second portion on the second region side is adjacent to the first portion on the third region side in the extending direction of the weft yarn, and
wherein the warp yarn is composed of a multifilament yarn.

8. The artificial blood vessel according to claim 7, wherein the first portion on the second region side and the first portion on the third region side are configured to extend continuously in an extending direction of the warp yarn in a zigzag shape.

9. The artificial blood vessel according to claim 6, wherein a curvature at the top part of the mountain part is less than a curvature at the bottom part of the valley part.

10. A manufacturing method for the artificial blood vessel according to claim 6,
the manufacturing method comprising the steps of:
preparing a tubular body configured by a weaving structure of the warp yarn and the weft yarn;
arranging the tubular body outside of a core material for molding, wherein the core material for molding has convex parts and concave parts corresponding to the mountain parts and the valley parts, respectively;
in a state in which the tubular body is arranged outside of the core material for molding, winding a winding member onto outside of the tubular body around a part of the tubular body in the circumferential direction along the concave parts of the core material for molding;
in a state in which the winding member is wound around the tubular body, relatively rotating, for a predetermined amount, a side in the axial direction of the tubular body, on which side the winding member is not wound around, relative to a side on which the winding member is wound around; and
firing the tubular body in which the mountain parts and the valley parts are formed by the winding member.
